(19) United States
(12) Patent Application Publication
(10) Pub. No.: US 2023/0270109 A1
(43) Pub. Date: Aug. 31, 2023

(54) DRY CONTACT DISINFECTANT

(71) Applicant: Game Shield Industries Inc., Calgary (CA)

(72) Inventors: Kyle Greene, Carmangay (CA); Jason Clemett, Calgary (CA); Jackson Clemett, Calgary (CA); Leslie McTavish, Lethbridge (CA)

(21) Appl. No.: 18/014,461

(22) PCT Filed: May 31, 2021
(86) PCT No.: PCT/CA2021/050739

§ 371 Date: Jan. 4, 2023

Related U.S. Application Data
(60) Provisional application No. 63/032,585, filed on May 30, 2020.

(51) Int. Cl. A01N 41/06; A01N 25/14; A01N 33/12; A01P 1/00; A61K 8/46; A61K 8/41; A61Q 17/00

(52) U.S. Cl. CPC A01N 41/06; A01N 25/14; A01N 33/12; A01P 1/00; A61K 8/466; A61K 8/416; A61Q 17/005

(57) ABSTRACT

A dry contact disinfectant includes a mixture of a powdered carrier and a powdered disinfectant. The powdered carrier has beneficial properties for a predetermined activity, and the powdered disinfectant is effective for killing one or more predetermined pathogens.

### TECHNICAL FIELD

This relates to a dry contact disinfectant, and in particular, a dry contact disinfectant suitable for use in disinfecting hands or other parts of a person during an activity.

### BACKGROUND

Many types of infectious agents, such as pathogenic bacteria, viruses or fungi, are transmitted by common surfaces that are touched by different people such as doors, handles, levers, doorknobs, light switches, handrails, elevator buttons, touch screens, keyboards and computer mice, coffeepot handles, countertops, drinking fountains, and other items that may be frequently touched by different people. Oftentimes, the transfer happens by hand contact, either by touching a common surface, or by hand-shaking, etc. As such frequent hand washing, and washing the common surfaces regularly is encouraged to reduce the risk of transmission.

### SUMMARY

According to an aspect, there is provided a dry contact disinfectant, comprising a mixture of a powdered carrier and a powdered disinfectant, the powdered carrier having beneficial properties for a predetermined activity, and the powdered disinfectant being effective for killing one or more predetermined pathogens.

According to other aspects, there is provided one or more of the following features, alone or in combination: the powdered disinfectant may be effective in a dry state and a wet state; the powdered carrier may comprise a drying agent; the powdered carrier may comprise a friction agent; the powdered carrier may comprise chalk, rosin, talc, sodium bicarbonate, or combinations thereof; the powdered disinfectant may comprises chloramine-T, benzalkonium chloride, or combinations thereof; the mixture may comprise 0.01%-0.1% w/w chloramine-T, 0.01%-0.1% w/w benzalkonium chloride, and 99.8-99.98% w/w sodium bicarbonate; a particle size of the powdered carrier may be within an order of magnitude of a particle size of the powdered disinfectant; there may be a sufficient concentration of powdered disinfectant to disinfect a surface while substantially maintaining the beneficial properties of the powdered carrier; there may be a container that contains the mixture, the container comprising a porous bag or an open container; and the mixture may be compressed to form a solid.

According to an aspect, there is provided a method of applying disinfectant to a human body, comprising the steps of: providing a dry contact disinfectant comprising a mixture of a powdered carrier and a powdered disinfectant, the powdered carrier having beneficial properties for a predetermined activity, and the powdered disinfectant being effective for killing one or more predetermined pathogens; and applying the dry contact disinfectant to a surface of a human in a sufficient amount to disinfect a surface of the one or more predetermined pathogens.

According to other aspects, the method may comprise one or more of the following aspects, alone or in combination: the mixture may be compressed to form a solid, the mixture may be carried in an open container, or the mixture may be carried in a porous bag; the surface may be a portion of the skin of the human or an article of clothing or equipment worn by the human; the powdered disinfectant may be effective in a dry state and a wet state; the powdered carrier may comprise chalk, rosin, talc, sodium bicarbonate, or combinations thereof; the powdered disinfectant may comprise chloramine-T, benzalkonium chloride, or combinations thereof; the mixture may comprise 0.01%-0.1% w/w chloramine-T, 0.01%-0.1% w/w benzalkonium chloride, and 99.8-99.98% w/w sodium bicarbonate; there may be a sufficient concentration of powdered disinfectant to disinfect a surface while substantially maintaining the beneficial properties of the powdered carrier.

In other aspects, the features described above may be combined together in any reasonable combination as will be recognized by those skilled in the art.

### BRIEF DESCRIPTION OF THE DRAWINGS

These and other features will become more apparent from the following description in which reference is made to the appended drawings, the drawings are for the purpose of illustration only and are not intended to be in any way limiting, wherein:

FIG. 1 is a perspective view of a porous bag.

FIG. 2 is a perspective view of an open-topped bag.

FIG. 3 is a perspective view of a bar made from compressed powder.

### DETAILED DESCRIPTION OF PREFERRED EMBODIMENTS

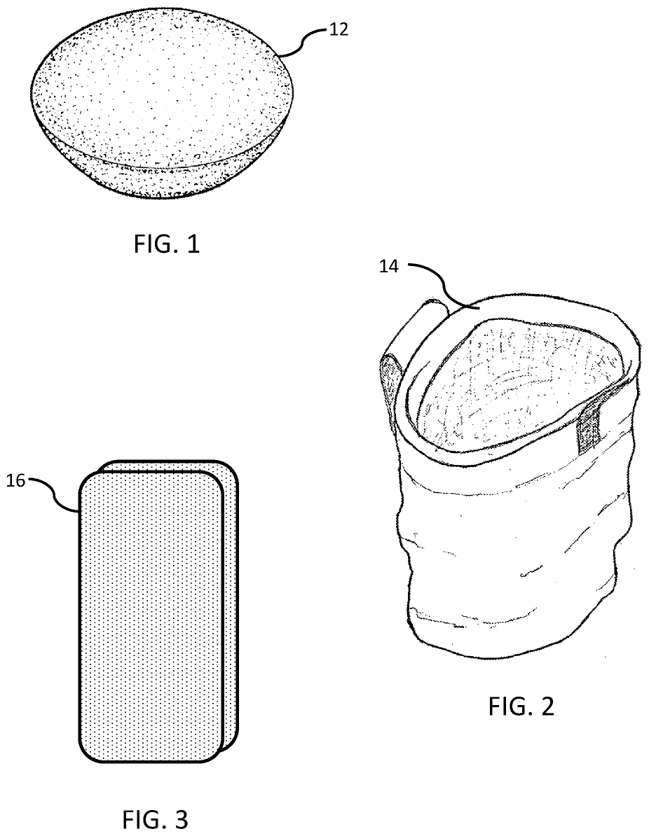

A dry contact disinfectant will now be described with reference to FIG. 1 through 3. The dry contact disinfectant includes a mixture of a carrier powder and a disinfectant powder combined in a suitable delivery method.

The carrier powder may be, for example, rosin, corn starch, talc, chalk, sodium bicarbonate, salt, etc., each of which may be intended for a specific purpose or activity. For example, some powders may be used to dry the hands, to increase the grip, etc., and specific powders may be preferable for specific activities. While it is anticipated that the dry contact disinfectant will be used primarily for hands, it will be understood that the dry contact disinfectant may be applied to other parts of the body, such as the feet, forearms, etc. that may also come into contact with others or shared surfaces.

The powdered disinfectant is a non-toxic disinfectant, which may be a biocide, selected to kill one or more desired pathogens, which may be a virus, bacteria and/or fungus. The disinfectant will be in a sufficient concentration to be effective, while being at a low enough concentration to minimally affect the properties of the carrier powder and not be harmful to a user's skin. As will be understood, the selected disinfectant may be a combination of different agents and may kill a broader range of pathogens than those that are specifically targeted. Examples of suitable disinfectants available in powdered form may be based on chloramine-T, such as Halamid™ or benzalkonium chloride. Each of these examples are typically used in solution, however it has been found that, when combined together in powdered form, may act as a strong disinfectant. Others powdered disinfectants may be used. Preferably, the disinfectant will have a particle size suitable for use with the carrier, such that they may be applied using the same delivery method and maintain an equal distribution within the carrier.

In one example, the dry contact disinfectant may include 0.01%-0.1% w/w chloramine-T, 0.01%-0.1% w/w benzalkonium chloride, and 99.8-99.98% w/w sodium bicarbonate, each in powdered form. In this example, sodium bicarbonate is the carrier powder, and acts as desiccant when the moisture level is sufficiently low, relative to the amount of dry contact desiccant applied, on the surface. It has been found that, at low moisture levels, benzalkonium chloride is less effective as a disinfecting agent, while chloramine-T is more effective and the desiccation properties of the sodium bicarbonate also help disinfect a surface. At high moisture levels, chloramine-T and sodium bicarbonate are less effective as disinfecting agents, while benzalkonium chloride is more effective. When used together, the disinfection capacity of the powdered disinfectant is improved, and is effective over a wider range of moisture levels than each component separately.

The ratio of powdered disinfectant to carrier powder may vary depending on various factors, such as the particle size of each, the dosage rate required (which in turn relates to the delivery method and the amount of carrier powder that would typically be applied), the size of surface being treated, etc. The ratio may also depend on whether the powdered disinfectant has a noticeable impact on the properties of the carrier powder, as the carrier powder will be intended to be used for a particular purpose, which in turn depends on how the powdered disinfectant is applied, such as by dipping, washing, sprinkling, etc.

During use, the dry contact disinfectant will be applied, for example, to the hands by a user. In some cases, the hands may be rubbed to “wash” the hands in the powder to distribute both the carrier powder and the disinfectant powder across the hands. The carrier powder will provide the necessary increase in grip or act to dry the hands, while the powdered disinfectant will disinfect the hands. If the carrier powder is dry and can absorb moisture, it may act as a secondary method to aid disinfection by dry killing the pathogen. Alternatively, depending on the type of carrier powder, the carrier powder may lift debris and dirt from skin as the powder is rubbed during application, which also helps clean the skin.

In addition to disinfecting the hands, the dry contact disinfectant may be applied directly to, or transferred to any surface being touched by the hands in which case it would provide some degree of secondary disinfection and further protect against transfer from a common surface. For example, a ball when handled by a player, a weight bar in the case of weight lifting, holds in the case of wall or rock climbing, tools or door handles in the case of workers, etc. may be protected from carrying pathogens as they are handled by a user with dry contact disinfectant on their hands. In some cases, moisture on the secondary surface (or the skin) may dissolve the disinfectant and also disinfect.

The dry contact disinfectant may be useful for primary and secondary surface disinfection and may be used in locations where liquid sanitizer or washing with soap and water is impractical or not useful, or where a common surface is continually contacted by many people and it is impractical or not possible to disinfect the surface after each touch. In addition, by including the powdered disinfectant in a carrier powder that is already used by participants in an activity, little or no changes in behaviour are required to disinfect hands and surfaces. The dry contact disinfectant may be used to help prevent transfer of pathogen from the person applying the powder and disinfecting the surface that the person may have touched.

In one example, in a sport such as baseball where the ball is being transferred between players continuously and the ball cannot be disinfected between contact with multiple players, each player may disinfect their hands using the dry contact disinfectant. This protects each player, and allows for secondary disinfection of the ball each time it is touched by the player. This may be accomplished, for example, with a rosin bag filled with the disinfectant powder and the carrying powder that is carried by each player in their pocket or in an easily accessible location, and periodically, such as after each play, the players would dose disinfectant onto their hands.

For other sports such as rock or wall climbing, bags of powdered chalk may be used to dry hands and help grip. This chalk may be held loose in a bag or in a porous bag that is bounced in a hand. When the climber uses the chalk, the powder disinfects their hands and is transferred to the grip. This prevents the climber from getting a pathogen, and also cleans the surface of the grip.

In another example, ballet dancers use solid rosin to help grip surfaces and other dancers, where the rosin may be a solid that transfers the powder to a surface, similar to a chalk stick. In this case, the powdered disinfectant and the rosin are homogenously mixed, and the mixture may be reformed under pressure into a solid stick or rock shape. The dose of the disinfectant is the layer of the rosin mixture transferred to the skin. This may be used to prevent the transfer of a pathogen from the dancer to another dancer to other equipment.

Referring to FIG. 1 a dosing bag 12 is shown. Dosing bag 12 is a porous or semi-permeable bag that contains the powder and applies the powder via its pores similar to other types of dosing bags, such as rosin bags used in baseball or the like. In particular, dosing bag 12 may have pores having a size that relates to the size of particles, in the powdered disinfectant and a desired dosing rate. In other examples, dosing bag 12 may be porous only on a portion of the surface, where a portion of the bag is impermeable. Other porous containers, or containers with porous surfaces such as a bottle with a porous lid, may also be used.

Referring to FIG. 2, an example of a powder bag 14 is shown. Powder bag 14 contains loose powder and is open at the top, where powder is applied by reaching into the bag. Powder bag 14 may be similar to known types of bags used, for example, for climbing. In this example, the dosing rate depends on the amount of powder that transfers to a user's hands, and the concentration of the disinfectant in the powder. In some examples, powder bag 14 may carry a dosing bag (not shown) within.

Referring to FIG. 3, an example of a transfer stick 16 is shown, depicted as a stick that has been formed by compressing the carrier powder and disinfectant powder into a solid. Transfer stick 16 is rubbed along skin or a surface and is designed to leave a residue. Alternatively, transfer stick 16 may be designed to be broken into pieces and crushed for use in, for example, bag 12 or 14.

In this patent document, the word “comprising” is used in its non-limiting sense to mean that items following the word are included, but items not specifically mentioned are not excluded. A reference to an element by the indefinite article “a” does not exclude the possibility that more than one of the elements is present, unless the context clearly requires that there be one and only one of the elements.

The scope of the following claims should not be limited by the preferred embodiments set forth in the examples above and in the drawings, but should be given the broadest interpretation consistent with the description as a whole.

1. A dry contact disinfectant, comprising:
a mixture of a powdered carrier and a powdered disinfectant, the powdered carrier having beneficial properties for a predetermined activity, and the powdered disinfectant being effective for killing one or more predetermined pathogens, the powdered disinfectant comprising a combination of chloramine-T and benzalkonium chloride.

2. The dry contact disinfectant of claim 1, wherein the powdered disinfectant is effective in a dry state and a wet state.

3. The dry contact disinfectant of claim 1, wherein the powdered carrier comprises a drying agent.

4. The dry contact disinfectant of claim 1, wherein the powdered carrier comprises a friction agent.

5. The dry contact disinfectant of claim 1, wherein the powdered carrier comprises chalk, rosin, talc, sodium bicarbonate, or combinations thereof.

6. (canceled)

7. The dry contact disinfectant of claim 1, wherein the mixture comprises 0.01%-0.1% w/w chloramine-T, 0.01%-0.1% w/w benzalkonium chloride, and 99.8-99.98% w/w sodium bicarbonate.

8. The dry contact disinfectant of claim 1, wherein a particle size of the powdered carrier is within an order of magnitude of a particle size of the powdered disinfectant.

9. The dry contact disinfectant of claim 1, comprising a sufficient concentration of powdered disinfectant to disinfect a surface while substantially maintaining the beneficial properties of the powdered carrier.

10. The dry contact disinfectant of claim 1, further comprising a container that contains the mixture, the container comprising a porous bag or an open container.

11. The dry contact disinfectant of claim 1, wherein the mixture is compressed to form a solid.

12. A method of applying disinfectant to a human body, comprising the steps of:
providing a dry contact disinfectant comprising a mixture of a powdered carrier and a powdered disinfectant, the powdered carrier having beneficial properties for a predetermined activity, and the powdered disinfectant being effective for killing one or more predetermined pathogens, the powdered disinfectant comprising a combination of chloramine-T and benzalkonium chloride; and
applying the dry contact disinfectant to a surface of a human in a sufficient amount to disinfect a surface of the one or more predetermined pathogens.

13. The method of claim 11, wherein mixture is compressed to form a solid, the mixture is carried in an open container, or the mixture is carried in a porous bag.

14. The method of claim 11, wherein the surface is a portion of skin of the human body or an article of clothing or equipment worn by the human body.

15. The method of claim 11, wherein the powdered disinfectant is effective in a dry state and a wet state.

16. (canceled)

17. The method of claim 11, wherein the mixture comprises 0.01%-0.1% w/w chloramine-T, 0.01%-0.1% w/w benzalkonium chloride, and 99.8-99.98% w/w sodium bicarbonate.

18. The method of claim 11, comprising a sufficient concentration of powdered disinfectant to disinfect a surface while substantially maintaining the beneficial properties of the powdered carrier.
